(19) United States
(12) Patent Application Publication
(10) Pub. No.: US 2016/0151437 A1
(43) Pub. Date: Jun. 2, 2016

(54) NUTRITIONAL SUPPLEMENTS FOR IMPROVED VISION AND METHODS OF TREATING THE SAME

(71) Applicant: Viva Pharmaceutical, Inc.

(72) Inventors: Jason Jiang-Chung, Richmond (CA); Xueju Xie, Langley (CA)

(73) Assignee: Viva Pharmaceutical, Inc.

(21) Appl. No.: 14/292,962

(22) Filed: Jun. 2, 2014

(51) Int. Cl. A61K 36/45; A61K 45/06; A61K 31/015; A61K 31/341; A61K 33/30; A61K 31/045

(52) U.S. Cl. CPC A61K 36/45; A61K 33/30; A61K 31/045; A61K 31/015; A61K 31/341; A61K 45/06

(57) ABSTRACT

A nutritional supplement for improving vision in a human is disclosed where the supplement contains bilberry fruit extract, lutein, zeaxanthin, beta carotene, and an omega-3.

A nutritional supplement for improving vision in a human is disclosed where the supplement contains bilberry fruit extract, lutein, zeaxanthin, beta carotene, an omega-3, black currant extract, zinc, selenium, vitamin B₁, vitamin B₂, vitamin B₆ and vitamin B₁₂.

A method for treating vision impairment is disclosed, the method of administering to a patient in need a nutritional supplement that contains a therapeutic amount of bilberry fruit extract, lutein, zeaxanthin, beta carotene, and an omega-3.

### FIELD

The present disclosure generally relates to nutritional supplements for improving vision.

### BACKGROUND

Antioxidants and free-radical scavenging compounds have numerous crucial biological functions in mammals especially when it comes to eye health.

Moreover, in adult humans, certain antioxidants, free-radical scavenging compounds, B-Vitamins, and Omega-3s have been linked to potentially promoting improved vision, night vision, and recovery after prolonged exposure to video display terminals and have been theorized to prevent or treat age-related macular degeneration (AMD), cataracts, and Retinitis pigmentosa (RP).

### SUMMARY

A nutritional supplement for improving vision in a human is disclosed where the supplement contains bilberry fruit extract, lutein, zeaxanthin, beta carotene, and an omega-3.

A nutritional supplement for improving vision in a human is disclosed where the supplement contains bilberry fruit extract, lutein, zeaxanthin, beta carotene, an omega-3, thiamine hydrochloride, riboflavin, vitamin B₆, and cyanocobalamin.

A method for treating vision impairment is disclosed, the method of administering to a patient in need a nutritional supplement that contains a therapeutic amount of bilberry fruit extract, lutein, zeaxanthin, beta carotene, an omega-3, calcuim ascorbate, L-lysine, selenium chelate, zinc citrate, and rutin.

### DETAILED DESCRIPTION

The present disclosure will now be described more fully hereinafter with reference to the accompanying drawings, in which some, but not all embodiments of the disclosure are shown. Indeed this disclosure may be embodied in many different forms and should not be construed as limited to the embodiments set forth hereinafter; rather, these embodiments are provided so that this disclosure will satisfy applicable legal requirements. Like numbers refer to like elements throughout.

In the last couple decades, many people have been suffering from eye strain or like symptoms resulting from working long hours on personal computers, video display terminals, and like equipment. Other visual problems include eye discomfort, blurring of distant objects, and asthenopia (visual fatigue).

Several dietary supplements have been shown to alleviate or reduce symptoms.

Examples of these are carotenoids, long-chain polyunsaturated fatty acids, and anthocyanosides.

Macular Degeneration (AMD)

Age-related macular degeneration (AMD) is the leading cause of blindness and irreversible vision loss (20/200 or worse) among people in the United States aged 52 or older. AMD in the most common overall cause of blindness in the United States. As people continue to rely on visual display terminals, AMD is expected to increase within the population. AMD is a degenerative disease of the macula, the area of the retina that is responsible for central vision and color perception. AMD tends to become worse with time and can best be described as a process of “wear and tear.” Thus, the prevalence of severe visual loss increases with age. AMD encompasses several types of abnormalities that develop in the macula of older people. These abnormalities range from mild to severe. Mild abnormalities usually are present with no loss of vision and severe abnormalities may result with loss of all straight-ahead vision. Because the peripheral retina is unaffected by AMD, side vision is retained along with the ability to see in the dark. Most affected is the ability to see fine detail, to read, and to see well enough in the distance to drive.

The macula is the part of the retina that lies directly behind the lens. The cones are light-sensitive cells that are responsible for central vision and are heavily concentrated in the macula. The peripheral retina is composed mainly of rods, which are the light-sensitive cells responsible for side and night vision. The macula is one hundred times more sensitive to detail that the peripheral retina. In a healthy macula, the clear layer of the retina on the inside of the eye is nourished and maintained by an adjoining layer called the pigment epithelium. Behind the pigment epithelium is the choroid that contains the blood vessels that transport nourishment to and carry waste material away from the retina.

A higher dietary intake of lutein, zeaxanthin, omega-3 long chain polyunsaturated fatty acids (docosahexaenoic acid [DHA] and eicosapentaenoic acid [EPA]), or both are associated with a decreased risk of developing advanced AMD.

Cataracts

A cataract is a clouding of the lens inside the eye. This leads to a decrease in vision. Cataracts are the most common cause of blindness and are conventionally treated with surgery. Visual loss occurs because the opacification of the lens obstructs light from passing and being focused on to the retina at the back of the eye. Cataracts are most commonly due to aging, however there are many other causes including genetics, trauma, genetics, skin disease, radiation, drug use, and medications.

Retinitis Pigmentosa

Retinitis pigmentosa (RP) is an inherited, degenerative eye disease that causes serious vision impairment and often blindness. The progression of RP is not consistent. Some people will exhibit symptoms from youth and others may not develop or notice symptoms until later in life. Generally, the later the onset, the more rapid is the deterioration in sight.

A form of retinal dystrophy, RP is caused by abnormalities of the photoreceptors (rods and cones) or the retinal pigment epithelium (RPE) of the retina leading to progressive sight loss. Affected individuals may experience defective light to dark, dark to light adaptation or nyctalopia (night blindness), as the result of the degeneration of the peripheral visual field (known as tunnel vision). Sometimes, central vision is lost first causing the person to look sidelong at objects. While there is no cure for retinitis pigmentosa, progression of the disease has been reduced through daily supplementation of beta-carotene.

Keratoconus

Keratoconus is a degenerative disease of the eye where structural changes within the cornea cause it to become thinner and change to a more conical shape than its normal gradual curve. Keratoconus is a genetic disease consisting in a non-inflammatory progressive dystrophy affecting approximately 50 people in every 100,000 each year—generally young people between 10 and 20 years of age. As disease frequency is higher amongst females, keratoconus etiology appears to be correlated to dysfunctions of endocrine glands (hypophysis and thyroid). In about 85% of cases, it can affect both eyes, and may have an evolution that may vary from subject to subject.

Upon onset of this disease, an irregular curvature appears that modifies the refractive power of the cornea, producing distortions of images and a confused close and distant vision. The patient complains in any case of a reduction of vision, above all distant vision. The vision continues to regress irreversibly, with a consequent need for frequent change of glasses, and may be mistaken for a myopia associated to astigmatism.

While many of the vision impairments listed above do not have permanent cures, treatment has been shown to be effective by administering therapeutic amounts of nutritional supplements. The following compounds have been effective in alleviating, slowing, and sometimes reversing these impairments. Additionally, these vitamins, minerals, and compounds act synergistically in improving vision. In the present disclosure, different formulations of nutritional supplements are disclosed that are effective in treating vision impairment.

Bilberry Fruit Extract

Bilberries include several closely related species of the Vaccinium genus. Bilberries contain nutrients, including many types of vitamins. In addition, clinical tests conducted mainly by public organizations in European countries such as Italy, France, and Germany have confirmed that a variety of anthocyanin salts included in bilberries are effective for relaxing eye strain.

Anthocyanins are water-soluble glycosides, which are flavylium cation derivatives.

Fifteen different anthocyanins are found in bilberry. Anthocyanins are known for their pharmacological effects such as improvement of visual functions, oxygen-scavenging activity in the body, free-radical scavenging, strengthening of blood vessels, anti-inflammatory effects, and antiulcer effects. Anthocyanins also help improve night vision and prevent cataracts and glaucoma.

The enhanced visual acuity at night provided by Anthocyanins is thought to be due to the acceleration of rhodopsin regeneration and/or activation of retinal enzymes. Oxidative stress destroys tissues in various diseases including neurodegenerative diseases such as amyotrophic lateral sclerosis, cancers, and cardiovascular diseases. Ocular disease, including age-related macular degeneration, is also a result of oxidative stress caused by the overproduction of reactive oxygen species (ROS). As the antioxidant, anthocyanin-rich bilberry extract, might have a tissue-protecting effect, it may have pharmaceutical application in human preventive therapy.

Anthocyanins may prevent myopic refractory shifts after visual tasks on video display terminals. Anthocyanins also promote recovery of visual acuity in cases of pseudomyopia in primary school students. Dietary anthocyanins serve to prevent myopic refractory shift during visual tasks and promoting visual recovery.

Additionally, anthocyanin salts contained in bilberries are usually highly absorbent in the human body. Also, eating bilberries or drinking bilberry juice in a usual manner can hardly attain ingestion of bilberry anthocyanin salts in an amount proven effective, or required, for relaxing eye strain as determined by clinical tests. Extracts of billberry fruit contain high amounts of anthocyanin salts. For nutritional supplements, Bilberry Fruit Extract should be 15%-50% anthocyanidins, in one embodiment, the Bilberry Fruit Extract is 25%. Bilberry Fruit Extract may be provided in nutritional supplements in differing amounts ranging from 20 mg to 200 mg.

Lutein

Lutein is a xanthophyll and one of 600 known naturally occurring carotenoids. Lutein is synthesized by plants, and like other xanthophylls is found in high quantities in green leafy vegetables such as kale, spinach, and also yellow carrots. In green plants, xanthophylls act to modulate light energy and serve as non-photochemical quenching agents to deal with triplet chlorophyll (an excited form of chlorophyll), which is overproduced at very high light levels, during photosynthesis.

Lutein is a lipophilic molecule and is generally insoluble in water. The presence of the long chromophore of conjugated double bonds (polyene chain) provides the distinctive light-absorbing properties. The polyene chain is susceptible to oxidative degradation by light or heat and is chemically unstable in acids.

Lutein (3,3′-dihydroxy-β,ε-carotene) represents an oxygenated carotenoid or xanthophyll. The physiological properties of lutein and particularly its function as an antioxidant are due to its potential to inactivate singlet oxygen and to quench active radicals.

Lutein is concentrated in the macula, a small area of the retina responsible for central vision. Lutein and zeaxanthin are the main components of the macular pigment. The hypothesis for the natural concentration is that lutein helps keep the eyes safe from oxidative stress and the high-energy photons of blue light. Various research studies have shown that a direct relationship exists between lutein intake and pigmentation in the eye.

Lutein may be obtained by animals directly or indirectly, from plants. Animals use lutein as an antioxidant and for blue light absorption. Lutein, together with zeaxanthin, is an essential component of the macular pigment in the retina of the eye. A low level of intake of this particular carotenoid increases the risk of age-related macular degeneration (AMD) and cataracts, which are the leading causes of visual impairment and acquired blindness, and are key quality of life issues among millions of ageing people. Consuming lutein and zeaxanthin was associated with a reduced risk of developing AMD.

Lutein is a natural part of human diet when fruits and vegetables are consumed. For individuals lacking sufficient lutein intake, lutein-fortified foods are available, or in the case of elderly people with a poorly absorbing digestive system, a sublingual spray is available. As early as 1996, lutein has been incorporated into dietary supplements.

Administration of lutein has shown to reduce the risk of developing advanced AMD. While no recommended daily allowance currently exists for lutein as for other nutrients, positive effects have been seen at dietary intake levels of 6-10 mg/day. Some embodiments provide 25 mg of lutein.

Lutein may be provided in a nutritional supplement in differing amounts ranging from 0.1 mg to 50 mg.

Zeaxanthin

Zeaxanthin is one of the most common carotenoid alcohols found in nature. It is the pigment that gives paprika (made from bell peppers), corn, saffron, wolfberries, and many other plants their characteristic color.

Zeaxanthin is a xanthophylls that is found in highest quantity in the leaves of most green plants, where they act to modulate light energy and perhaps serve as a non-photochemical quenching agent to deal with triplet chlorophyll (an excited form of chlorophyll), which is overproduced at very high light levels, during photosynthesis.

Like lutein, animals derive zeaxanthin from a plant diet. Zeaxanthin is one of the two primary xanthophyll carotenoids contained within the retina of the eye. Within the central macula, zeaxanthin is the dominant component, whereas in the peripheral retina, lutein predominates.

Zeaxanthin supplements may be used to treat different disorders, mainly with affecting the eyes. Zeaxanthin focused on the retina of the center of the macula, clear single molecular oxygen produced by ultraviolet rays cause damage to the eyes, can selectively accumulate in the eye for macular and provide macular pigment, thereby improving eye health, the protective effect of the visual.

Zeaxanthin also has been reported to have a potent antioxidant function and an anti-tumor effect. Further, zeaxanthin is known to be, together with lutein, present in the retina and crystalline lens and involved in the maintenance of eye health. Due to these physiological effects, zeaxanthin is useful as a material for health foods, cosmetics, or pharmaceuticals.

Several observational studies have connected high dietary intake of foods providing zeaxanthin with lower incidence of age-related macular degeneration (AMD), most notably the Age-Related Eye Disease study (AREDS). Suggests that supplementation with lutein and zeaxanthin can help improve human vision at low light levels. Benefits from supplementation may be more apparent in subpopulations of individuals exposed to high oxidative stress, such as heavy smokers or those with poor nutrition.

Zeaxanthin may be provided in a nutritional supplement in differing amounts ranging from 1 mg to 50 mg.

Astaxanthin

Astaxanthin belongs to a class of naturally-occurring pigments called carotenoids and belongs to a larger class of phytochemicals known as terpenes, which are built from five carbon precursors; isopentenyl diphosphate and dimethylallyl diphosphate. Astaxanthin is found in microalgae, yeast, salmon, trout, hill, shrimp, crayfish, crustaceans, and the feathers of some birds. It provides the red color of salmon meat and the red color of cooked shellfish.

Astaxanthin potentially enhances endurance and recovery. Astaxanthin has a positive effect is on the mitochondria because it is a powerful antioxidant, and astaxanthin has been shown to effectively scavenge free radicals from muscle tissue and help reduce singlet oxygen. Astaxanthin is able to cross the blood-brain barrier, while beta-carotene and some other antioxidants don't, and protects the brain and nervous system from oxidative stress.

Research shows that, due to astaxanthin's potent antioxidant activity, it may be beneficial in ocular, cardiovascular, immune, inflammatory and neurodegenerative diseases. Some research supports the assumption that it may protect body tissues from oxidative and ultraviolet damage through its suppression of NF-κB activation.

Astaxanthin may be provided in a nutritional supplement in differing amounts ranging from 1 mg to 20 mg.

Black Currant Extract (Anthocyanidin/Anthocyanoside)

Black currant fruits and juice are known to be rich in anthocaynosides. Like bilberry fruit extract that contain high amounts of anthocyanin, black currant extract contains high amounts of anthocyanosides. The distinction between anthocyanins and anthocyanosides is that anthocyanins do not have the sugar content associated with anthocyano sides.

One concern about anthocyanins and anthocyanosides is that these healthy elements are easily lost if cooked or stored. For this reason, those who really want to get the optimal health benefit from a diet of fresh produce will make sure they get their fruits and vegetables as fresh as possible. This is also why nutritional supplementation is helpful.

Studies have shown that an increased intake of black current anthocyanosides affect the rod components in the eye that are involved in dark adaption, which may help improve night vision. Additionally, black currant extract has had the effect of preventing myopic refractory shift after visual tasks on video display terminals. Supplementing a patient's nutrition with a nutritional supplement containing black currant extract will help patients eyes recover from viewing prolonged video display terminals, as visual fatigue is improved with such supplementation. Visual recovery is enhanced by dietary supplementation of anthocyanosides due to their function of improving dark adaption and rhodopsin regeneration.

Black currant extract may be provided in a nutritional supplement in differing amounts ranging from 10 mg to 100 mg.

β-carotene and Vitamin A

Vitamin A is a group of unsaturated nutritional organic compounds, that includes retinol, retinal, retinoic acid, and several provitamin A carotenoids, among which beta-carotene is the most important. β-Carotene is a strongly colored red-orange pigment abundant in plants and fruits. It is an organic compound and chemically is classified as a hydrocarbon and specifically as a terpenoid (isoprenoid), reflecting its derivation from isoprene units. β-Carotene is the substance in carrots, pumpkins, and sweet potatoes that colors them orange and is the most common form of carotene in plants.

β-Carotene has been used to treat various disorders such as erythropoietic protoporphyria. It has also been used to reduce the risk of breast cancer in women before menopause, and the risk of age-related macular degeneration (AMD).

The role of vitamin A in the visual cycle is specifically related to the retinal form. Within the eye, 11-cis-retinal is bound to protein “opsin” to form rhodospin in rods and iodopsin (cones) at conserved lysine residues. As light enters the eye, the 11-cis-retinal is isomerized to the all-“trans” form. The all-“trans” retinal dissociates from the opsin in a series of steps called photo-bleaching. This isomerization induces a nervous signal along the optic nerve to the visual center of the brain. After separating from opsin, the all-“trans”-retinal is recycled and converted back to the 11-“cis”-retinal form by a series of enzymatic reactions. In addition, some of the all-“trans” retinal may be converted to all-“trans” retinol form and then transported with an interphotoreceptor retinol-binding protein (IRBP) to the pigment epithelial cells. Further esterification into all-“trans” retinyl esters allow for storage of all-trans-retinol within the pigment epithelial cells to be reused when needed. The final stage is conversion of 11-cis-retinal will rebind to opsin to reform rhodopsin (visual purple) in the retina. Rhodopsin is needed to see in low light (contrast) as well as for night vision. Kühne showed that the regeneration of rhodopsin only occurs when retina is attached to retinal pigmented epithelial (RPE). It is for this reason that a deficiency in vitamin A will inhibit the reformation of rhodopsin and lead to one of the first symptoms, night blindness.

Increasing the availability of carotinoids, and particularly beta-carotene, to the retinal pigment epithelium, function can be normalized. In fact, it has been suspected for some time that carotinoids are present in the human eye as reflected by the term macula lutea, lutea meaning yellow.

In the treatment of age related macular degeneration or in the prevention or improvement of impaired vision in an eye with drusen, beta-carotene is administered in a therapeutically effective amount. Therapeutically effective amounts of beta-carotene are those amounts sufficient to stabilize the progression of the disease or to resolve the symptoms of ARMD. This amount will depend upon the age, weight, sex, sensitivity, and the like of the individual. In many mammals, the therapeutically effective amount can be determined by experimentation well known in the art such as by establishing a matrix of dosages and frequencies and assigning a group of experimental subjects to each point in the matrix.

Typically for a human being, that amount of dietary supplementation will be at least about 50 mg/day of beta-carotene. Most preferably, that amount will range from about 1 mg/day to about 350 mg/day.

β-Carotene may be provided in differing amounts ranging from 1 mg to 20 mg.

Thiamine Hydrochloride (Vitamin B₁)

Thiamine is a water-soluble vitamin of the B complex. Animals must obtain it from their diet, and thus, for them, it is an essential nutrient. Well-known syndromes caused by thiamine deficiency include beriberi, Wernicke-Korsakoff syndrome, and optic neuropathy. Optic neuropathy can also occur in thiamine deficiency and is characterized by bilateral visual loss, cecocentral scotomas and impaired color perception. The ophthalmological findings usually can show a bilateral oedema of the optic disk in the acute phase, followed by a bilateral optic atrophy.

Thiamine is found in a wide variety of foods at low concentrations. Yeast, yeast extract, and pork are the most highly concentrated sources of thiamine. In general, cereal grains are the most important dietary sources of thiamine, by virtue of their ubiquity.

Thiamine may be provided in differing amounts ranging from 0.07 mg to 100 mg.

Riboflavin (Vitamin B₂)

Riboflavin, also known as vitamin B₂ is an easily absorbed colored micronutrient with a key role in maintaining health in humans and other animals. It is the central component of the cofactors FAD and FMN, and is therefore required by all flavoproteins. As such, vitamin B₂ is required for a wide variety of cellular processes. It plays a key role in energy metabolism, and for the metabolism of fats, ketone bodies, carbohydrates, and proteins.

Flavin mononucleotide (FMN) and flavin adenine dinucleotide (FAD) function as coenzymes for a wide variety of oxidative enzymes and remain bound to the enzymes during the oxidation-reduction reactions. Flavins can act as oxidizing agents because of their ability to accept a pair of hydrogen atoms. Reduction of isoalloxazine ring (FAD, FMN oxidized form) yields the reduced forms of the flavoproteins (FMNH2 and FADH2).

Flavoproteins play important roles in the electron transport chain. Fatty acyl CoA dehydrogenase requires FAD in fatty acid oxidation. FAD is required to convert retinol (vitamin A) to retinoic acid. Reduction of the oxidized form of glutathione (GSSG) to its reduced form (GSH) is also FAD dependent.

Riboflavin has been used in a new treatment to slow or stop the progression of the corneal disorder keratoconus. This is called corneal collagen crosslinking (CXL). In corneal crosslinking, riboflavin drops are applied to the patient's corneal surface. Once the riboflavin has penetrated through the cornea, ultraviolet A light therapy is applied. This induces collagen crosslinking, which increases the tensile strength of the cornea. The treatment has been shown in several studies to stabilize keratoconus.

Some people use riboflavin for eye conditions including eye fatigue, cataracts, and glaucoma.

Riboflavin may be provided in differing amounts ranging from 0.08 mg to 100 mg.

Vitamin B₆ (Pyridoxine)

Vitamin B₆ is widely distributed in foods in both its free and bound forms. Good sources include vegetables, nuts, meats, whole grain products, and bananas. Cooking, storage, and processing may cause a loss of vitamin B₆ and in some foods more than 50% may be lost, depending on the form of vitamin present in the food. Plant foods lose the least during processing, as they contain mostly pyridoxine, which is far more stable than the pyridoxal or pyridoxamine found in animal foods.

Pyridoxine is required for the production of the monoamine neurotransmitters serotonin, dopamine, norepinephrine and epinephrine, as it is the precursor to pyridoxal phosphate: cofactor for the enzyme aromatic amino acid decarboxylase. This enzyme is responsible for converting the precursors 5-hydroxytryptophan (5-HTP) into serotonin and melatonin, and levodopa (L-DOPA) into dopamine, noradrenaline and adrenaline. As such it has been implicated in the treatment of depression and anxiety.

Mental depression is another condition which may result from low vitamin B₆ intake. Because of pyridoxine's role in serotonin and other neurotransmitter production, supplementation often helps depressed people feel better, and their moods improve significantly. Some studies suggest the vitamin B₆-magnesium combination can also help attention deficit disorder, citing improvements in hyperactivity, hyperemotivity/aggressiveness and improved school attention. Poor vitamin B₆ status has been hypothesized to play a role in the cognitive decline that some older adults experience.

Vitamin B₆ deficiency is associated with conjunctivitis, which is inflammation of the conjunctiva (the outermost layer of the eye and the inner surface of the eyelids).

Vitamin B₆ is usually safe, at regular intakes up to 200 mg per day in adults.

However, vitamin B₆ can cause neurological disorders, such as loss of sensation in legs and imbalance, when taken in high doses (200 mg or more per day-10,000% of US RDA) over a long period of time. Vitamin B₆ toxicity can damage sensory nerves, leading to numbness in the hands and feet as well as difficulty walking. Symptoms of a pyridoxine overdose may include poor coordination, staggering, numbness, decreased sensation to touch, temperature, and vibration, and tiredness for up to six months.

Pyridoxine may be provided in nutritional supplements in differing amounts ranging from 0.1 mg to 10 mg.

Cyanocobalamin (Vitamin B₁₂)

Cyanocobalamin is a naturally occurring member of the vitamin B₁₂ family. Cobalamin is required for two important reactions: the conversion of methylmalonyl CoA to succinyl CoA, a Krebs cycle intermediate, and the conversion of homocysteine to methionine, a reaction in which the methyl group of methyltetrahydrofolate is donated to remethylate homocysteine. Cobalamin deficiency, even in the absence of hematologic signs, may lead to impaired cognitive performance in adolescents. Decreased cobalamin status is strongly associated with cognitive dysfunction in the elderly.

Cyanocobalamin may be provided in differing amounts ranging from 0.14 μg to 1000 μg.

Omega-3s

Polyunsaturated fatty acids have numerous crucial biological functions in mammals. For example, in humans, numerous polyunsaturated fatty acids have been linked to cell membrane synthesis, metabolism, and maintenance. Moreover, in adult humans, certain polyunsaturated fatty acids have been linked to potentially promoting improved cognitive ability and have been theorized as potential treatments for arthritis, cancer, diabetes, lupus, and psoriasis. Also, certain polyunsaturated fatty acids have been linked to promoting retinal and brain development in fetuses and newborns.

For example, docosahexanoic acid is one such polyunsaturated fatty acid that has been implicated in playing a role in fetus and newborn development as well as potentially playing a role in maintaining and/or promoting improved human cognitive ability.

Omega-3 fatty acids refer to a group of three fats called ALA (found in plant oils), EPA, and DHA (both commonly found in marine oils). Common sources of animal omega-3 EPA and DHA fatty acids include fish oils, egg oil, squid oils, hill oil, while some plant oils contain the omega 3 ALA fatty acid such as seabuckthorn seed and berry oils, clary sage seed oil, algal oil, flaxseed oil, Sacha Inchi oil, Echium oil, and hemp oil.

Omega-3 fatty acids, and DHA may be provided through many embodiments, including the sources listed above. A nutritional supplement containing Omega-3s may include EPA from 200 mg to 800 mg and DHA from 100 mg to 600 mg or a daily dose between 100 mg to 5,000 mg of DHA+EPA.

Some additives may be used in preserving the potency of the ingredients listed above.

A nonexclusive list examples are provided below.

Calcium Ascorbate

Vitamin C is necessary for optimal immune function and better health. As it is a water-soluble vitamin, the body does not store vitamin C and it must therefore be obtained through diet or other supplemental sources. As the body's primary antioxidant, it protects the aqueous areas of the body (including the blood, intracellular fluid and interstitial fluid) from free radicals. Vitamin C also helps the body heal wounds, repair and maintain cartilage, bones, teeth, and gums. Vitamin C protects LDL cholesterol from oxidation. Studies have shown certain conditions can benefit from supplemental vitamin C including diabetes, cataracts and heart disease due to is antioxidant effect. Some people use vitamin C for depression, thinking problems, dementia, Alzheimer's disease, physical and mental stress, fatigue, and attention deficit-hyperactivity disorder (ADHD). Vitamin C is also used for glaucoma and preventing cataracts.

Calcium Ascorbate is a natural form of Vitamin C, which is more readily absorbed in the blood stream than the other forms of calcium. Calcium Ascorbate is approved for use as a food additive. Some people find supplementing diets with regular vitamin C (ascorbic acid) can cause gastrointestinal problems such as abdominal pain, diarrhea, or heartburn. Non-Acidic Calcium Ascorbate Vitamin C is buffered with calcium, which helps prevent stomach irritation.

Calcium ascorbate may be provided in nutritional supplements in the amount from 25 mg to 300 mg.

L-Lysine

L-Lysine is an essential amino acid. Lysine is not synthesized in animals, hence it must be ingested as lysine or lysine-containing proteins.

Lysine has a known anxiolytic action through its effects on serotonin receptors in the intestinal tract. One study showed that overstimulation of the 5-HT4 receptors in the gut are associated with anxiety-induced intestinal pathology. Lysine, acting as a serotonin antagonist and therefore reducing the overactivity of these receptors, reduced signs of anxiety and anxiety-induced diarrhea in the sample population. Another study showed that lysine deficiency leads to a pathological increase in serotonin in the amygdala, a brain structure that is involved in emotional regulation and the stress response.

L-Lysine may be provided in nutritional supplements in the amounts from 20 mg to 200 mg.

Selenium

Selenium is an essential trace element that exerts its physiological role as selenocysteine residue in at least 25 distinct selenoenzymes in mammals. Lack of GPx-1 due to alimentary selenium deprivation has been inferred to induce cataracts. The role of other selenoproteins in the eye is not entirely known. Selenium in excess of the tiny amounts required for selenoprotein synthesis is toxic in general and causes cataracts in experimental animals. A protective role of selenium in the development of cataract, macula degeneration, retinitis pigmentosa or any other ocular disease is not definitely known but suspected.

Selenium may be provided as a dietary supplement as a chelate.

Selenium may be provided in the amounts from 20 mcg to 100 mcg.

Zinc

Zinc is an essential trace mineral. Zinc is believed to be important for vision because high levels of the mineral are found in the macula, part of the retina. It plays a vital role in bringing vitamin A from the liver to the retina in order to produce melanin, a protective pigment in the eyes. Zinc is highly concentrated in the eye, mostly in the retina and choroid, the vascular tissue layer lying under the retina. Impaired vision has been linked to zinc deficiency. A deficiency of zinc can result in poor night vision and cloudy cataracts. Some studies show that getting enough zinc can help you see better at night. Clinical studies demonstrate zinc supplementation can increase general wellness, and that it has a beneficial impact on connective tissue as well as reproductive health and eye health.

Zinc is recommended for individuals diagnosed as being at high-risk for age-related macular degeneration (AMD), or already experiencing the early stages of AMD. The human body does not synthesize the zinc it needs, which is the reason why red meat, seafood, poultry, eggs, wheat germ, mixed nuts, black-eyed peas, tofu, and baked beans are essential to good nutrition. Daily intake of zinc through diet, nutritional supplements, or fortified foods and beverages is important for the maintenance of good eye health.

Zinc is essential for growth, immune system function, testosterone metabolism, as a cofactor for the antioxidant enzyme superoxide dismutase, and for many other functions in the body. Zinc is involved in the functioning of over 200 enzymes and plays a key role in genetic expression, cell division, and growth. Zinc may be provided to a human in the form of zinc citrate.

Zinc deficiency has been associated with adverse impacts on a number of structures and functions of the body. Individuals susceptible to zinc deficiency include the elderly, the immuno-compromised, those on parenteral nutrition, and burn victims.

Low-molecular-weight organic acids, such as citrate, have a positive effect on zinc absorption. Certain dietary factors, such as phytic acid found in grains, can interfere with the body's absorption of minerals. An animal study found citrate can offset the negative effects of dietary phytic acid on zinc absorption.

Therefore, the present disclosure can improve eye blood circulation, protect the retina from ultraviolet visible damage, can effectively protect the eyes, the prevention and reduction of myopia, hyperopia, amblyopia is generated, and easy to use.

Zinc plays an important role in supporting the body's defense system, and is related to the normal absorption and actions of the B vitamins. Zinc promotes healthy skim, hair and nails, supports normal taste and vision, and promotes the synthesis of collagen in bone tissue. It also supports cell growth and DNA formation. It exerts antioxidant activity and can support a healthy immune system.

Zinc supplementation may be provided in the form of zinc citrate, in the form of a chelate, or in other forms.

Zinc may be provided in a nutritional supplement in an amount from 2 mg to 60 mg.

Vitamin E

The term “vitamin E” describes not just one substance, but a group of eight compounds called tocopherols and tocotrienols. Alpha-tocopherol is the most active form of vitamin E. Natural forms of vitamin E are designated with a “d-” prefix (d-alpha-tocopherol, for example); synthetic vitamin E has a “dl-” prefix (dl-alpha-tocopherol). Natural vitamin E (the “d” forms) is more beneficial for your eyes and body than synthetic (“dl”) vitamin E.

Vitamin E is a powerful antioxidant that helps protect membranes of cells throughout the body against damage caused by metabolic by-products called free radicals. Some studies suggest that E helps to possibly prevent cataracts, and it might be yet another factor in preventing macular degeneration (AMD).

Almonds and other nuts are excellent sources of natural vitamin E.

In the Age-Related Eye Disease Study (AREDS) involving nearly 5,000 people, researchers found a 25 percent lower risk of developing advanced stages of AMD when a nutritional formula including vitamin E was taken. The AREDS supplement included 400 International Units (IU) of vitamin E, as well as high levels of vitamin A (as beta-carotene), vitamin C, and zinc. Based on AREDS and other nutritional studies, many eye doctors recommend that their patients supplement their diet with a daily multivitamin that contains up to 400 IU of vitamin E in combination with other antioxidants as part of their preventative eye care.

Vitamin E may be provided in a nutritional supplement in an amount from 25 IU to 100 IU.

Rutin

Rutin is one of the phenolic compounds found in the invasive plant species Carpobrotus edulis and contributes to the antibacterial and antioxidant properties of the plant. Both quercetin and rutin are used in many countries as medications for blood vessel protection, and are ingredients of numerous multivitamin preparations and herbal remedies.

Rutin inhibits aldose reductase activity. Aldose reductase is an enzyme normally present in the eye and elsewhere in the body.

Rutin may be provided in a nutritional supplement in the amounts from 5 mg to 50 mg.

Vitamin B₃

Niacin is a form of Vitamin B₃. Vitamin B₃ is found in many foods including yeast, meat, fish, milk, eggs, green vegetables, beans, and cereal grains. Niacin is found in many vitamin B complex supplements with other B vitamins.

Niacin is used for high cholesterol. It is also used along with other treatments for circulation problems, migraine headache, dizziness, and to reduce the diarrhea associated with cholera. Niacin is also used for preventing positive urine drug screens in people who take illegal drugs. Niacin and niacinamide is used for preventing vitamin B₃ deficiency and related conditions such as pellagra. Each of these forms of vitamin B₃ is used for schizophrenia, hallucinations due to drugs, Alzheimer's disease and age-related loss of thinking skills, chronic brain syndrome, depression, motion sickness, alcohol dependence, and fluid collection (edema).

Some people use niacin or niacinamide for acne, leprosy, attention deficit-hyperactivity disorder (ADHD), memory loss, arthritis, preventing premenstrual headache, improving digestion, protecting against toxins and pollutants, reducing the effects of aging, lowering blood pressure, improving circulation, promoting relaxation, improving orgasm, and preventing cataracts.

Vitamin B₃ may be provided in a nutritional supplement in the amounts from 5 mg to 60 mg.

Gou Qi Zi concentrate (Fructus Lycii, wolfberry)

Gou Qi Zi is also known as the Chinese Wolfberry Fruit or wolfberries. Wolfberries have a significant nutrient value and antioxidant content. Wolfberries contain many nutrients and phytochemicals. They have been termed a superfruit.

Gou Qi Zi concentrate is beneficial because it brightens the eyes, improves blood circulation to nourish eyes, prevent dizziness and blurred vision, and improves acuity. There are reported biological effects of wolfberries supplementation in animal models, and speculated from this basic research that there may be potential benefits against cardiovascular and inflammatory diseases, vision-related diseases (such as age-related macular degeneration and glaucoma.

Gouqizi concentrate may be provided in a nutritional supplement in the amounts from 5 mg to 60 mg.

Eyebright Concentrate

Eyebright is used in many aliments of the eye, as well as slowing down progressive weakness due to age. Eyebright can use to treat eye inflammation such as ophthalmia, blepharitis, cataracts, pink eye, and bloodshot eye, also can use to treat colds, sinusitis and seasonal allergies.

Although intake of the active ingredients present in the compositions of this disclosure are practically unrestricted, it may be adjusted according to the pharmaceutical dosage form and the user age, body weight, symptoms and general conditions. For example, a preferred daily dosage range is from 0.3 to 6.86 mg of anthocyanin per kg body weight, preferably, 0.35 to 3 mg, more preferably, 0.35 to 1.5 mg. In another example, a daily dose is in the range of 240-480 mg bilberry extract containing 25% anthocyanin, which is equal to 60 mg to 120 mg anthocyanin daily. Nutritional supplements may be formulated containing the ingredients disclosed above. They may be provided in the form of a tablet, capsule or soft gel, which methods of making are well known in the art. It also can be in the powder form and liquid form.

Nutritional supplements may be formulated containing the ingredients disclosed above. They may be provided in the form of a tablet, capsule or soft gel, which methods of making are well known in the art.

Dosages may be formulated for daily consumption and in some circumstances a nutritional supplement may need to be taken more than once daily.

When the compositions of the disclosure are used in solid form for the oral administration, a variety of pharmaceutically or physiologically acceptable carriers may be used, e.g. excipients such as lactose and glucose, binding agents such as water and ethanol, disintegrating agents such as powdered agar and dry starch, disintegration inhibitors such as stearin and cacao powder, adsorbing agents such as kaolin and bentonite, absorption promoters such as sodium lauryl sulfate, moisturizing agents such as glycerin and starch and lubricant agents such as powdered borate and polyethylene glycol.

In many instances, thiamine hydrochloride is administered in solid dosage forms, typically of tablets, either singly or in combination with one or more other vitamins and/or some other drug or drugs.

It should be emphasized that the embodiments described herein are merely possible examples of implementations, merely set forth for a clear understanding of the principles of the present disclosure. Many variations and modifications may be made to the described embodiment(s) without departing substantially from the spirit and principles of the present disclosure. Further, the scope of the present disclosure is intended to cover any and all combinations and sub-combinations of all elements, features, and aspects discussed above. All such modifications and variations are intended to be included herein within the scope of the present disclosure, and all possible claims to individual aspects or combinations of elements or steps are intended to be supported by the present disclosure.

One should note that conditional language, such as, among others, “can,” “could,” “might,” or “may,” unless specifically stated otherwise, or otherwise understood within the context as used, is generally intended to convey that certain embodiments include, while alternative embodiments do not include, certain features, elements and/or steps. Thus, such conditional language is not generally intended to imply that features, elements and/or steps are in any way required for one or more particular embodiments or that one or more particular embodiments necessarily include logic for deciding, with or without user input or prompting, whether these features, elements and/or steps are included or are to be performed in any particular embodiment. Unless stated otherwise, it should not be assumed that multiple features, embodiments, solutions, or elements address the same or related problems or needs.

Various implementations described in the present disclosure may include additional systems, methods, features, and advantages, which may not necessarily be expressly disclosed herein but will be apparent to one of ordinary skill in the art upon examination of the following detailed description and accompanying drawings. It is intended that all such systems, methods, features, and advantages be included within the present disclosure and protected by the accompanying claims.

1. A nutritional supplement for a human, the supplement comprising: bilberry fruit extract, lutein, zeaxanthin, beta carotene, and an omega-3.

2. The nutritional supplement of claim 1, additionally comprising: astaxanthin, and black currant extract (anthocyano side) and vitamin C.

3. The nutritional supplement of claim 2, additionally comprising: zinc, selenium, vitamin B1, vitamin B2, vitamin B₆, and vitamin B12

4. The nutritional supplement of claim 3, additionally comprising: L-lysine, and rutin.

5. The nutritional supplement of claim 1, wherein the bilberry fruit extract is in the amount of from 50 mg to 1000 mg, lutein is in the amount of from 3 mg to 60 mg, zeaxanthin is in the amount of from 0.2 mg to 8 mg, beta carotene is in the amount of from, and an omega-3 is in the amount of from 0.05 mg to 6 g.

6. The nutritional supplement of claim 1, wherein the bilberry fruit extract is in the amount of 80 mg, lutein is in the amount of 20 mg, zeaxanthin is in the amount of 2 mg, beta carotene is in the amount of 8,000 IU, and an omega-3 is in the amount of 600 mg

7. The nutritional supplement of claim 2, wherein astaxanthin is in the amount of from 0.1 mg to 50 mg, anthocyanoside is in the amount of from 1 mg to 400 mg and vitamin C is in the amount of from 10 mg to 1000 mg.

8. The nutritional supplement of claim 2, wherein astaxanthin is in the amount of 4 mg, anthocyanoside is in the amount of 40 mg, and vitamin C is in the amount of 150 mg.

9. The nutritional supplement of claim 3, wherein the vitamin B₁ is in the amount of from 1 mg to 100 mg, vitamin B₂ is in the amount of from 1 mg to 100 mg, vitamin B₆ is in the amount of from 1 mg to 100 mg, and vitamin B₁₂ is in the amount of from 0.1 μg to 1500 μg.

10. The nutritional supplement of claim 3, wherein the vitamin B₁ is in the amount of 2 mg, vitamin B₂ is in the amount of 2 mg, vitamin B₆ is in the amount of 2 mg, and vitamin B₁₂ is in the amount of 0.1 mg.

11. A method for treating vision impairment, the method comprising: administering to a patient in need a nutritional supplement comprising a therapeutic amount of bilberry fruit extract, lutein, zeaxanthin, beta carotene, and an omega-3.

12. The method of claim 10, wherein the nutritional supplement additionally comprises:
astaxanthin, black currant extract (anthocyanoside), vitamin C, zinc, selenium, vitamin B₁, vitamin B₂, vitamin B₆, and vitamin B₁₂.

13. A nutritional supplement consisting essentially of:
Lutein, Zeaxanthin, Astaxanthin, Black currant extract (anthocyanoside), Vitamin B2, Vitamin B3, Vitamin C, Zinc, Omega 3s, Vitamin E, Bilberry Extract (Anthocyanidins), Selenium, Beta Carotene, Gou Qi Zi concentrate (Fructus Lycii, wolfberry), Eyebright concentrate, Vitamin B1, Vitamin B6, Vitamin B12, Vitamin A, L-Lysine, Rutin, Lutein, Zeaxanthin, Astaxanthin, Black currant extract (anthocyanoside), Vitamin B2, Vitamin B3, Vitamin C, Zinc, Omega-3s, Vitamin E, Bilberry Extract (Anthocyanidins), Selenium, Beta Carotene, Gouqizi concentrate (Fructus Lycii, wolfberry), Eyebright concentrate, Vitamin B1, Vitamin B6, Vitamin B12, Vitamin A, L-Lysine, and Rutin.
